(19) United States
(12) Patent Application Publication
(10) Pub. No.: US 2025/0195507 A1
(43) Pub. Date: Jun. 19, 2025

(54) Use of Pirfenidone (PFD) Combined With Nintedanib (BIBF 1120) in Preparation of Drug for Treating Pneumoconiosis

(71) Applicant: Institute of Basic Medical Sciences, CAMS, Beijing (CN)

(72) Inventors: Jing WANG, Beijing (CN); Meiyue SONG, Beijing (CN); Jiaxin WANG, Beijing (CN); Lu BAI, Beijing (CN)

(21) Appl. No.: 18/680,893

(22) Filed: May 31, 2024

(30) Foreign Application Priority Data
Dec. 19, 2023 (CN) 2023117546407

(51) Int. Cl. A61K 31/496; A61K 31/4418; A61K 47/38; A61P 11/00

(52) U.S. Cl. CPC A61K 31/496; A61K 31/4418; A61K 47/38; A61P 11/00

(57) ABSTRACT

Provided is use of pirfenidone (PFD) combined with nintedanib (BIBF 1120) in preparation of a drug for treating pneumoconiosis, which relates to the technical field of biomedicine. In the present disclosure, silicosis model mice in a fibrosis stage are used as a subject to allow research. A combined drug of PFD and BIBF 1120 to silicosis mice may effectively alleviate the progression of silicosis. The combined drug shows superiority in lung function, inflammation, and fibrosis, and exhibits an effect better than therapy with PFD or BIBF 1120 alone. Meanwhile, after the combined administration of PFD and BIBF 1120, there is no significant change in a body weight of the mice, and there is also no significant increase in liver function injury indicators such as serum aminotransferase, alanine aminotransferase (ALT), and aspartate aminotransferase (AST).

### CROSS REFERENCE TO RELATED APPLICATION

This patent application claims the benefit and priority of Chinese Patent Application No. 2023117546407 filed with the China National Intellectual Property Administration on Dec. 19, 2023, the disclosure of which is incorporated by reference herein in its entirety as part of the present application.

### REFERENCE TO SEQUENCE LISTING

A computer readable XML file entitled “GWP20240100808_seqlist”, that was created on Mar. 12, 2024, with a file size of about 13,750 bytes, contains the sequence listing for this application, has been filed with this application, and is hereby incorporated by reference in its entirety.

### TECHNICAL FIELD

The present disclosure belongs to the technical field of biomedicine, specifically relates to use of pirfenidone (PFD) combined with nintedanib (BIBF 1120) in preparation of a drug for treating pneumoconiosis.

### BACKGROUND

Pneumoconiosis is an occupational disease caused by workers' long-term exposure to large amounts of production dust, with lung inflammation and progressive fibrosis as the main pathological changes. Pneumoconiosis belongs to a global disease with high disability and mortality rates and seriously endangers human health. Once pneumoconiosis occurs, the remaining dust in the lungs continues to interact with alveolar macrophages. Even after the workers leave the dusty working environment, this disease continues to progress, manifesting as progressively worsening dyspnea and decreased lung function, eventually leading to death from respiratory failure. Unfortunately, there is still a lack of treatment to halt the progression of pneumoconiosis. In terms of non-drug treatment, whole lung alveolar lavage can only remove an extremely small amount of dust particles but cannot stop the progression of pneumoconiosis; while lung transplantation cannot be used as a routine treatment due to high surgical cost, lack of lung sources, and high risks. In terms of drug treatment, the efficacy and mechanism of a traditional drug tetrandrine is still unclear. Stem cell therapy has a certain potential in the treatment of pneumoconiosis, but its specific mechanism is not fully understood. Since the safety and effectiveness are still under study, the stem cell therapy currently cannot meet clinical demands. In addition, pneumoconiosis responds poorly to hormone treatment, which is closely related to the pathological characteristics of pneumoconiosis as shown by research reports. Facing the current situation that there is no drug available for the treatment of pneumoconiosis, it would greatly shorten the time and cost and improve the success rate to explore whether the existing clinical drugs for interstitial pulmonary diseases can be used for the treatment of silicosis.

Pirfenidone (PFD) and nintedanib (BIBF 1120) are globally approved for the treatment of idiopathic pulmonary fibrosis (IPF), and their anti-inflammatory and anti-fibrotic effects have been widely recognized. In multiple clinical trials, it is found that both of the above drugs can effectively improve lung function and prognosis of various interstitial lung diseases. PFD is a multi-effect pyridone compound with anti-fibrotic, anti-inflammatory, and antioxidant effects. In terms of anti-fibrosis, PFD reduces the production of profibrotic cytokines, chemokines, and growth factors, and inhibits fibroblast activation and collagen synthesis by weakening the TGF-β signaling pathway (including Smad3, p38, and Akt). In terms of anti-inflammation, PFD reduces inflammation mainly by reducing inflammatory cells. In terms of antioxidation, PFD can reduce key molecules of oxidative stress, including lipid peroxidation and advanced lipoxidation end products, and activities of superoxide dismutase and myeloperoxidase. BIBF 1120 is an oral small-molecule tyrosine kinase inhibitor that targets platelet-derived growth factor receptor (PDGFR), fibroblast growth factor receptor (FGFR), and vascular endothelial growth factor receptor (VEGFR), as well as non-receptor tyrosine kinases in the Src family of tyrosine kinases. In terms of anti-inflammation, BIBF 1120 can slow down T cell-mediated inflammation by inhibiting the activity of tyrosine kinase Lck, can reduce the release of chemokine CCL18 to inhibit the polarization of pro-fibrotic macrophages, and can inhibit the activation of monocytes. In terms of anti-fibrosis, BIBF 1120 inhibits the production of growth factors such as platelet-derived growth factor (PDGF), fibroblast growth factor (FGF), and vascular endothelial growth factor (VEGF), thereby inhibiting the proliferation of fibroblasts and transdifferentiation into myofibroblasts mediated by the above growth factors; in addition, BIBF 1120 can also reduce the expression of TGF-β and procollagen I. Therefore, it may provide new strategies for the clinical treatment of pneumoconiosis by exploring the effect and mechanism of PFD combined with BIBF 1120 in the treatment of pneumoconiosis.

### SUMMARY

In view of this, an objective of the present disclosure is to provide use of PFD and BIBF 1120 in preparation of a drug for treating pneumoconiosis. The combined administration of PFD and BIBF 1120 is effective in the treatment of pneumoconiosis, and half-dose combination and full-dose combination show equivalent efficacy, both of which are better than single drug.

To achieve the above objective, the present disclosure provides the following technical solutions:

The present disclosure provides use of PFD combined with BIBF 1120 in preparation of a drug for treating pneumoconiosis.

In some embodiments, the pneumoconiosis is one or more selected from the group consisting of silicosis, coal workers' pneumoconiosis (CWP), graphite pneumoconiosis, carbon black pneumoconiosis, asbestosis, talc pneumoconiosis, cement pneumoconiosis, mica pneumoconiosis, kaolin pneumoconiosis, aluminosis, arc-welders' pneumoconiosis (AWP), and foundry workers' pneumoconiosis (FWP).

In some embodiments, a PFD solution has a concentration of 10 mg/mL to 30 mg/mL.

In some embodiments, a BIBF 1120 solution has a concentration of 2 mg/mL to 5 mg/mL.

In some embodiments, the PFD solution or the BIBF 1120 solution is prepared with 1% carboxymethylcellulose sodium (CMC-Na) as a solvent.

The present disclosure further provides a combined drug for treating pneumoconiosis, including PFD and BIBF 1120.

In some embodiments, PFD and BIBF 1120 are at a mass concentration ratio of 5 to 15:1.

In some embodiments, the combined drug further includes a pharmaceutically acceptable carrier.

In some embodiments, the carrier includes CMC-Na.

In some embodiments, a dosage form of the combined drug is selected from the group consisting of a powder, a tablet, a capsule, and a solution.

Compared with the prior art, the embodiments of the present disclosure have the following beneficial effects:

The present disclosure provides use of PFD and BIBF 1120 in preparation of a drug for treating pneumoconiosis. In the present disclosure, silicosis model mice are used as a subject to allow research, and the combined administration of PFD+BIBF 1120 to silicosis mice may effectively alleviate the progression of silicosis; moreover, the combined administration of high and low doses shows an equivalent efficacy. Specifically, after combined administration of high and low doses of PFD+BIBF 1120, the lung function of silicosis mice is significantly improved, and there is no significant difference between the two doses, including in lung volume indicators such as inspiratory capacity (IC), forced vital capacity (FVC), and forced expiratory volume in one hundred seconds (FEV100), as well as in pulmonary ventilation function tests such as inspiratory resistance (RI), dynamic compliance (Cdyn), quasi-static compliance (Cchord), and maximum mid-expiratory flow (MMEF). The concentrations of inflammatory factors IL-1β and IL-6 in the alveolar lavage fluid of silicosis mice, the transcription levels of inflammatory factors Il-1β, Il-6, and Tnf-α in lung tissue, pulmonary inflammatory exudation, the transcription and expression levels of fibrosis factors FN-1 and Col-I, the collagen-specific amino acid hydroxyproline, and the fibrosis lesions are reduced, and the degree of lesions is alleviated. Moreover, combination therapy with PFD+BIBF 1120 shows superiority in terms of pulmonary function, inflammation, and fibrosis, and exhibits a better effect than therapy with PFD or BIBF 1120 alone. Meanwhile, after the combined administration of PFD+BIBF 1120, there is no significant change in a body weight of the mice, and there is also no significant increase in liver function injury indicators such as serum aminotransferase, alanine aminotransferase (ALT), and aspartate aminotransferase (AST). According to the above results, the combined treatment of PFD and BIBF 1120 has a desirable safety profile. Therefore, administration of PFD+BIBF 1120 may be used as a new therapeutic regimen for pneumoconiosis.

### BRIEF DESCRIPTION OF THE DRAWINGS

FIGS. 1A-1H show the results of combined administration of PFD+BIBF 1120 in improving lung function in silicosis mice; where FIG. 1A is a schematic diagram of the experiment of combined administration of PFD+BIBF 1120 to treat silicosis mice, FIG. 1B shows mouse IC, and FIG. 1C shows FVC; FIG. 1D shows FEV100; FIG. 1E shows MMEF, FIG. 1F shows RI, FIG. 1G shows Cdyn, FIG. 1H shows Cchord; *P<0.05, **P<0.01, ***P<0.001, #P<0.05, ##P<0.01, ###P<0.001;

FIGS. 2A-2G show the results of combined administration of PFD+BIBF 1120 in reducing inflammation in silicosis mice; where FIG. 2A is HE staining of mouse lung tissue, FIG. 2B shows Szapiel score, FIG. 2C shows the transcription level of Il-1β in mouse lung tissue, FIG. 2D shows the concentration of IL-1β in mouse bronchoalveolar lavage fluid, FIG. 2E shows the transcription level of Il-6 in mouse lung tissue, FIG. 2F shows the concentration of IL-6 in mouse bronchoalveolar lavage fluid, and FIG. 2G shows the transcription level of Tnf-α in mouse lung tissue; *P<0.05, **P<0.01, ***P<0.001, #P<0.05, ##P<0.01, ###P<0.001; and

FIGS. 3A-3K show the results of combined administration of PFD+BIBF 1120 in reducing fibrosis in silicosis mice; where FIG. 3A shows Masson staining of mouse lung tissue, FIG. 3B shows pulmonary fibrosis score, FIG. 3C shows the transcription level of Fn-1 in mouse lung tissue, FIG. 3D shows the transcription level of Col-I in mouse lung tissue, FIG. 3E shows a Western blot (WB) electrophoresis of FN-1 and COL-I, FIG. 3F is a graph showing the statistics of FN-1 of mouse lung tissue in the WB experiment, FIG. 3G is a graph showing the statistics of COL-I of mouse lung tissue in the WB experiment, FIG. 3H shows the hydroxyproline content in mouse lung tissue, FIG. 3I shows the mouse body weight change curve, FIG. 3J shows the mouse serum aminotransferase ALT content, and FIG. 3K shows the mouse serum aminotransferase AST content; *P<0.05, **P<0.01, ***P<0.001, #P<0.05, ##P<0.01, ###P<0.001.

### DETAILED DESCRIPTION OF THE EMBODIMENTS

The present disclosure provides use of PFD combined with BIBF 1120 in preparation of a drug for treating pneumoconiosis.

Types of the pneumoconiosis are not particularly limited in the present disclosure, and all types of the pneumoconiosis may be diagnosed based on the “diagnostic criteria for pneumoconiosis” and “pathological diagnostic criteria for pneumoconiosis”, and fall within the protection scope of the present disclosure, preferably including silicosis, CWP, graphite pneumoconiosis, carbon black pneumoconiosis, asbestosis, talc pneumoconiosis, cement pneumoconiosis, mica pneumoconiosis, kaolin pneumoconiosis, aluminosis, AWP, and FWP.

In the present disclosure, a PFD solution has a concentration of preferably 10 to 30 mg/mL, more preferably 15 to 25 mg/mL. In a specific example, the PFD solution has a concentration of preferably 10 mg/mL, 15 mg/mL, 20 mg/mL, 25 mg/mL, 30 mg/mL, and 35 mg/mL.

In the present disclosure, a BIBF 1120 solution has a concentration of preferably 2 to 5 mg/mL, more preferably 2.5 to 4 mg/mL. In a specific example, the BIBF 1120 solution has a concentration of preferably 2.5 mg/mL, 3 mg/mL, 3.5 mg/mL, 4 mg/mL, 4.5 mg/mL, and 5 mg/mL.

In the present disclosure, the PFD solution or the BIBF 1120 solution is prepared with 1% CMC-Na as a solvent. In a specific example, the PFD solution uses CMC-Na as a solvent, and has a concentration of preferably 30 mg/mL; the BIBF 1120 solution uses CMC-Na as a solvent, and has a concentration of preferably 5 mg/mL; a low dose for combined administration is preferably a suspension of 15 mg/mL PFD and 2.5 mg/mL BIBF 1120 suspended in CMC-Na; a high dose for combined administration is preferably a suspension of 30 mg/mL PFD and 5 mg/mL BIBF 1120 suspended in CMC-Na.

The present disclosure further provides a combined drug for treating pneumoconiosis, including an effective dose of PFD and an effective dose of BIBF 1120.

In some embodiments, PFD and BIBF 1120 are at a mass concentration ratio of preferably 5 to 15:1; more preferably 6 to 10:1. In a specific example, when the silicosis model mice in the fibrosis stage are treated by enteral administration, the PFD solution is enterally administered at a dose of 360 mg/kg once a day for 4 weeks; the BIBF 1120 solution is enterally administered at a dose of 60 mg/kg once a day for 4 weeks; the low-dose co-administration solution is administered by gavage at the dose of PFD 180 mg/kg+BIBF 1120 30 mg/kg, once a day for 4 weeks; the high-dose co-administration solution is administered by gavage at the dose of PFD 360 mg/kg+BIBF 1120 60 mg/kg, once a day for 4 weeks.

In the present disclosure, the combined drug further includes a pharmaceutically acceptable carrier.

In the present invention, the carrier includes but is not limited to CMC-Na.

In the present invention, the dosage form of the combined drug is not particularly limited, and preferably selected from the group consisting of a powder, a tablet, a capsule, and a solution.

The technical solution provided by the present disclosure will be described in detail below with reference to the examples, but they should not be construed as limiting the claimed scope of the present disclosure.

### Example 1

The combined administration of PFD+BIBF 1120 was effective in treating silicosis, with an effect better than that of a single drug.

### Construction of a Mouse Model of Silicosis and Administration of PFD+BIBF 1120

Male C57BL/6J mice (aged 8 weeks and weighing 25-30 g) were selected and housed in a specific pathogen free (SPF) facility for laboratory animal, and the silicosis model was established by one-time intratracheal instillation of silica (Si); the mice were divided into 5 groups (n=9):

(1) Si+Vehicle group: a silica suspension (300 mg/mL) was injected into the trachea with 40 μL, and a same amount of 1% CMC-Na was administered by gavage 6 weeks later, 1 time a day, 7 d a week, for 4 weeks.

(2) Si+PFD group: a silica suspension (300 mg/mL) was injected into the trachea with 40 μL, and PFD (prepared with 1% CMC-Na, 30 mg/mL) 360 mg/kg was administered by gavage 6 weeks later, 1 time a day, 7 d a week, for 4 weeks.

(3) Si+BIBF 1120 group: a silica suspension (300 mg/mL) was injected into the trachea with 40 μL, and BIBF 1120 (prepared with 1% CMC-Na, 5 mg/mL) 60 mg/kg was administered by gavage 6 weeks later, 1 time a day, 7 d a week, for 4 weeks.

(4) Si+Low combination (PFD+BIBF 1120) group: a silica suspension (300 mg/mL) was injected into the trachea with 40 μL, and PFD 180 mg/kg+BIBF 1120 30 mg/kg (prepared with 1% CMC-Na) was administered by gavage 6 weeks later, 1 time a day, 7 d a week, for 4 weeks.

(5) Si+High combination (PFD+BIBF 1120) group: a silica suspension (300 mg/mL) was injected into the trachea with 40 μL, and PFD 360 mg/kg+BIBF 1120 60 mg/kg (prepared with 1% CMC-Na) was administered by gavage 6 weeks later, 1 time a day, 7 d a week, for 4 weeks. All mice were sacrificed after 10 weeks, and the corresponding samples were collected for detection.

### Pulmonary Function Test

Anesthetized mice were fixed on an experimental table, and mouse lung function was detected by a pulmonary function testing system (DSI Buxco, USA). Before experiment, the mice were anesthetized by intraperitoneal injection of 0.4 mL/100 g 2% pentobarbital, tracheotomy was performed, a trachea cannula was inserted, and a ventilator was connected. Next, functional residual capacity (FRC), pressure-volume (PV) loop, flow-volume (FV) loop, and residual capacity (RC) were automatically assessed by a pulmonary function test (PET) system. Indicators closely related to lung function changes in silicosis were selected for statistical analysis, including lung volume indicators such as IC, FVC, and FEV100, and pulmonary ventilation function tests such as RI, Cdyn, Cchord, and MMEF.

### Pathological Staining

The left lung was fixed in 4% paraformaldehyde for 72 h, dehydrated, paraffin-embedded, sectioned (to 5 μm), and subjected to HE staining and Masson staining, respectively. The sections were scanned, photographed and counted by a 3D HISTECH digital slide scanner. The HE staining was conducted to calculate the inflammation score based on Szapiel's score. The scoring method included no inflammation (grade 0), mild (grade 1), moderate (grade 2), and severe (grade 3); Masson staining was conducted to evaluate the degree of fibrosis based on the King's score. Specifically, different silicosis nodules were evaluated for fibrosis degree according to King's method, with levels ranging from 0 to 5. The fibrosis level score (0-5) of each silica nodule was multiplied by its percentage of a total area of the tissue section.

### Enzyme-Linked Immunosorbent Assay (ELISA)

Concentrations of the inflammatory factors IL-1β and IL-6 in mouse bronchoalveolar lavage fluid (BALF) were detected by ELISA kits.

### Western Blot

Lung tissue proteins of all mice were extracted. A 10% polyacrylamide gel was prepared, and 10 μg of protein was loaded and electrophoresed at 80 V for 20 min; after the voltage was adjusted to 120 V, the electrophoresis was continued until it ended. Polyvinylidene fluoride (PVDF) membrane was used for the transfer at 260 mA for 1.5 h and blocked with 5% skimmed milk powder for 1 h, the primary antibody FN-1 (abcam, ab2413, 1:1000) was incubated at 4° C. overnight and the secondary antibody was incubated at room temperature for 1 h, and finally blots were developed by chemiluminescence. An 8% polyacrylamide gel was prepared with the same sample loading and electrophoresis conditions as before. Nitrocellulose (NC) membrane was used for the transfer at 220 mA for 2 h and blocked with 5% skimmed milk powder for 1 h, the primary antibody COL-I (abcam, ab254113, 1:1000) was incubated at 4° C. overnight and the secondary antibody was incubated at room temperature for 1 h, and finally blots were developed by chemiluminescence. The above bands were incubated with 8-actin primary antibody as internal control, and the remaining steps were identical as described above.

Quantitative Polymerase Chain Reaction (qPCR)

Lung tissue RNA of all mice was extracted; cDNA was obtained by using a reverse transcription kit (KR103, Tiangen Biotechnology, Beijing, China); qPCR was conducted by using a SYBR Green I Q-PCR Kit (TransGen Biotech, Beijing China); data collection and analysis were conducted by Bio-Rad IQ5 system.

TABLE 1
Primer sequences (5′ to 3′)

| | | | |
|---|---|---|---|
| β-actin | F | TAGGCACCAGGGTGTGAT | SEQ ID NO: 1 |
| | R | CTCCTCAGGGGCCACA | SEQ ID NO: 2 |
| Fn-1 | F | GACGAAGAGCCCTTACAGTTCCA | SEQ ID NO: 3 |
| | R | TCTGCAGTGCCTCCACTATG | SEQ ID NO: 4 |
| Col-I | F | CCTGGTCCCTCTGGAAATG | SEQ ID NO: 5 |
| | R | GGAAGCCTCTTTCTCCTCTC | SEQ ID NO: 6 |
| Il-1β | F | CAAGCTTCCTTGTGCAAGTGTC | SEQ ID NO: 7 |
| | R | TTCATCTTTTGGGGTCCGTCA | SEQ ID NO: 8 |
| Il-6 | F | TTCCTCTCTGCAAGAGACTTC | SEQ ID NO: 9 |
| | R | GTTGGGAGTGGTATCCTCTG | SEQ ID NO: 10 |
| Tnf-α | F | AGACACCATGAGCACAGAAA | SEQ ID NO: 11 |
| | R | CACTTGGTGGTTTGTGAGTG | SEQ ID NO: 12 |

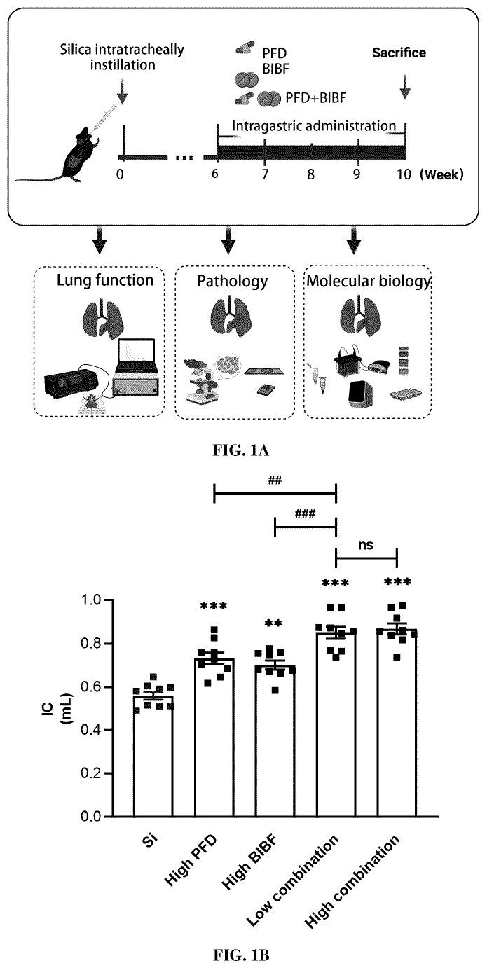

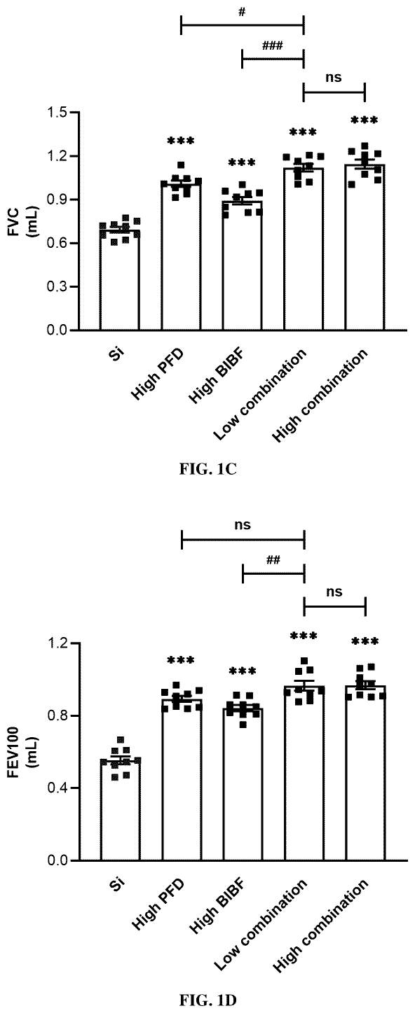

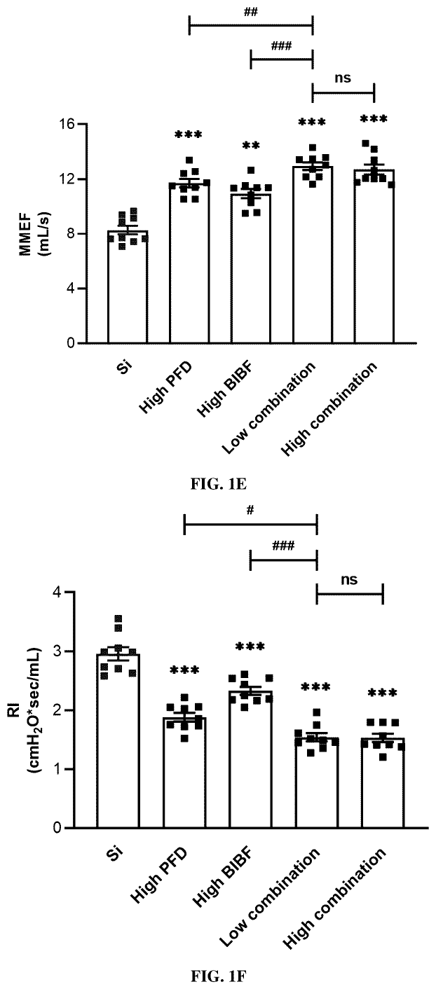

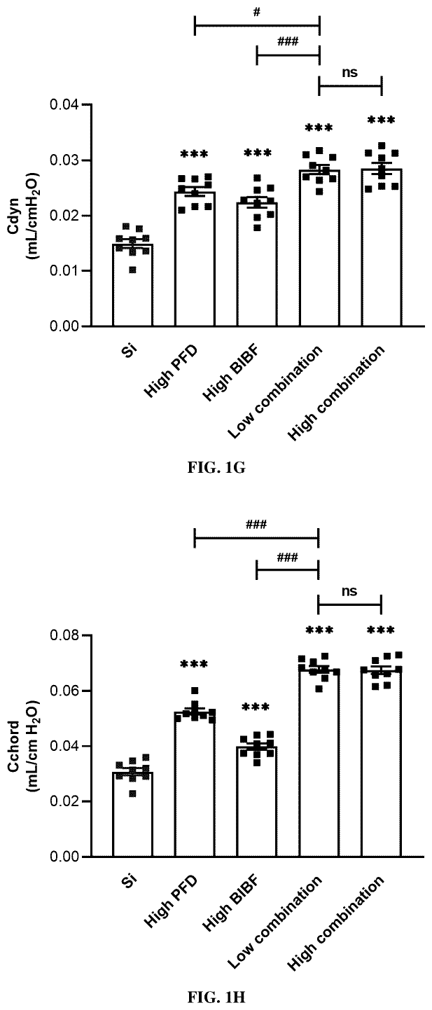

As shown in FIGS. 1A-1E, the combined administration of PFD+BIBF 1120 to silicosis mice could effectively alleviate the progression of silicosis, and there was no significant difference between high and low doses. After the combined administration of PFD+BIBF 1120, the lung function of silicosis mice was significantly improved, including lung volume indicators such as enhanced IC (FIG. 1B), increased FVC (FIG. 1C), and increased FEV100 (FIG. 1D), as well as pulmonary ventilation function tests such as increased RI (FIG. 1F), increased Cdyn (FIG. 1G), increased Cchord (FIG. 1H), and increased MMEF (FIG. 1E).

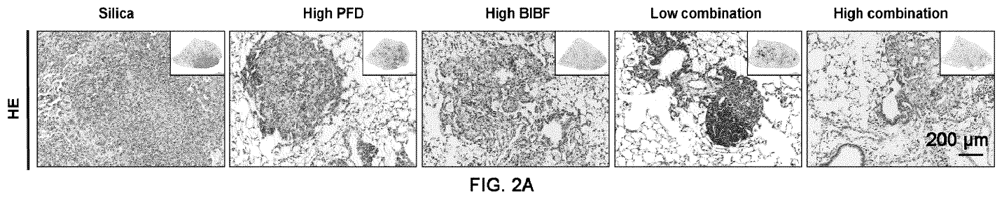

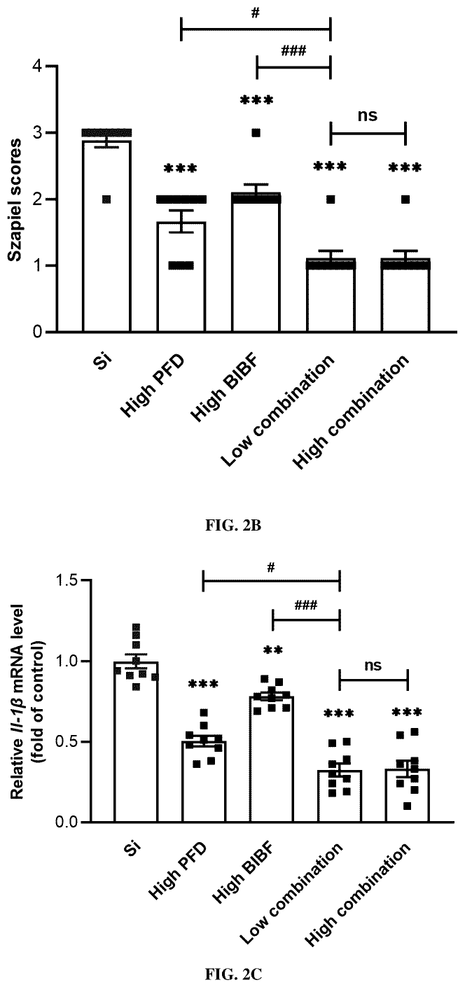

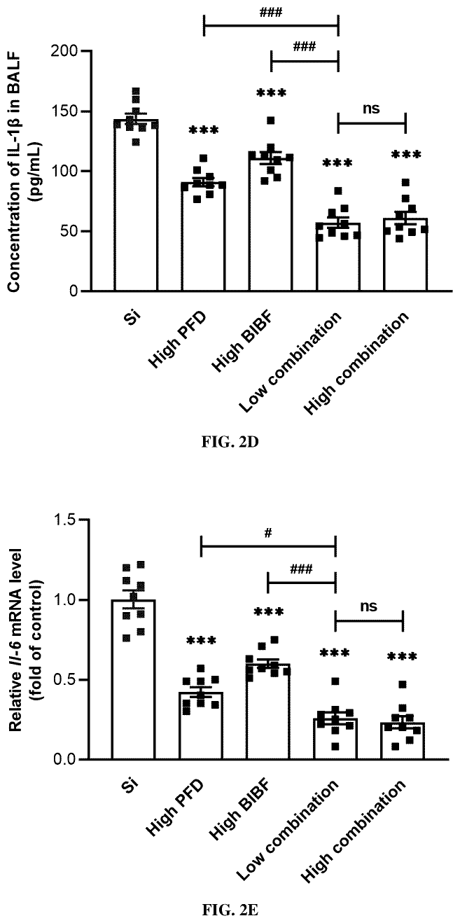

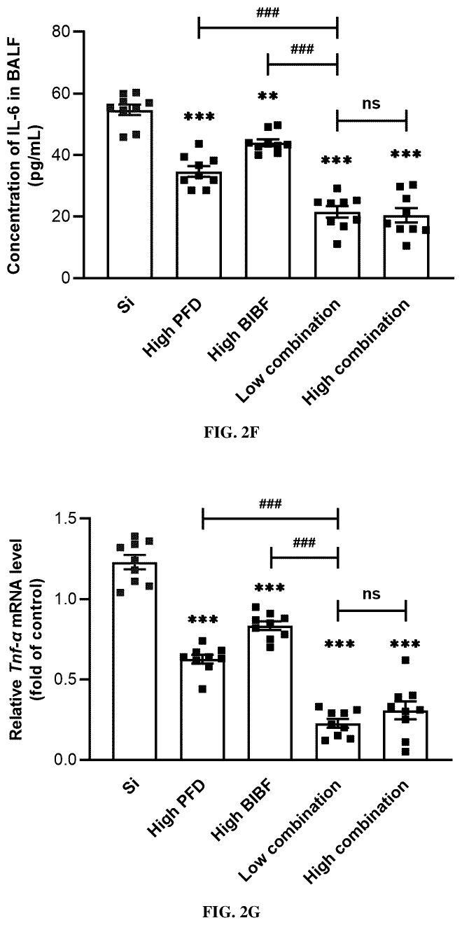

According to the results in FIGS. 2A-2G, after the combined administration of PFD+BIBF 1120: inflammatory exudation in the lungs of mice was reduced (FIG. 2A and FIG. 2B); concentrations of inflammatory factors IL-1β (FIG. 2D) and IL-6 (FIG. 2F) in the alveolar lavage fluid of silicosis mice were decreased; and transcription levels of inflammatory factors Il-1β (FIG. 2C), Il-6 (FIG. 2E), and Tnf-α (FIG. 2G) were reduced in lung tissue.

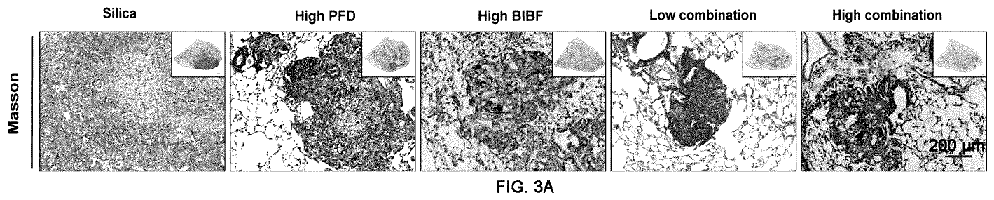

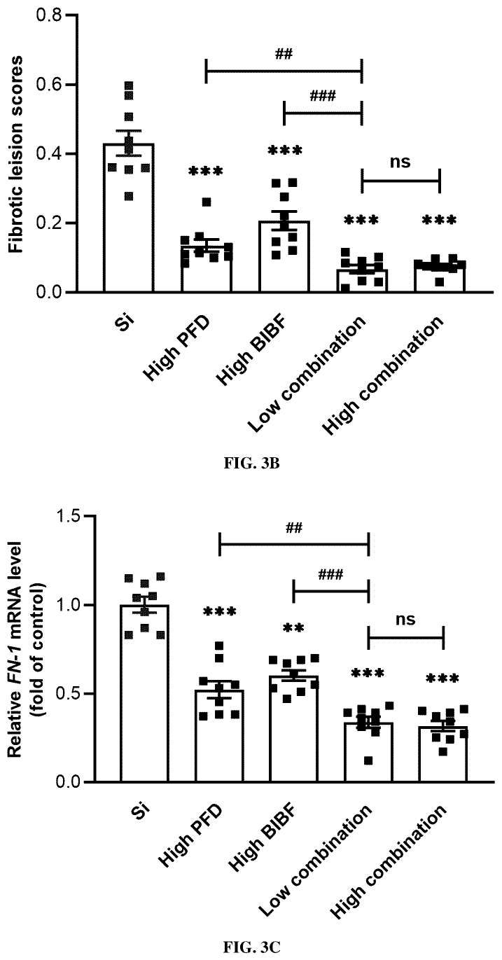

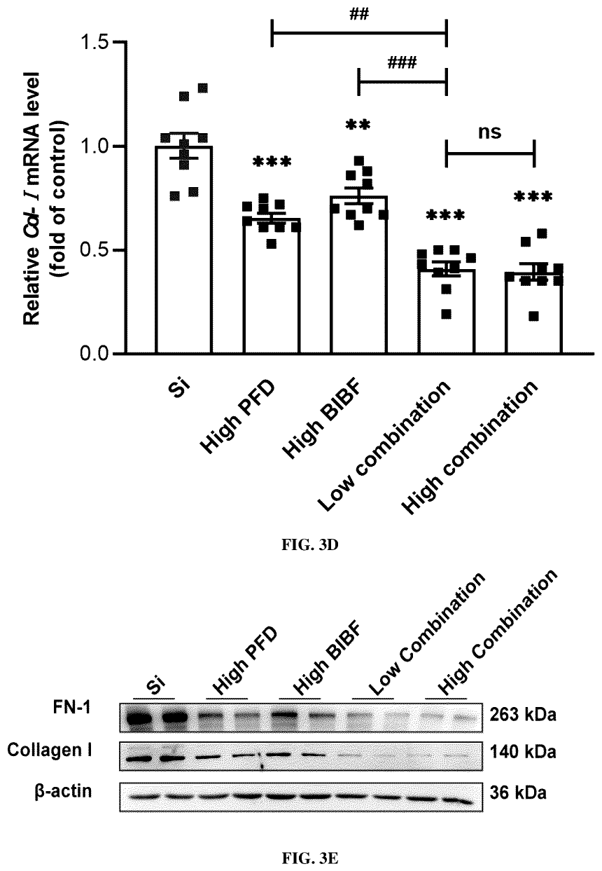

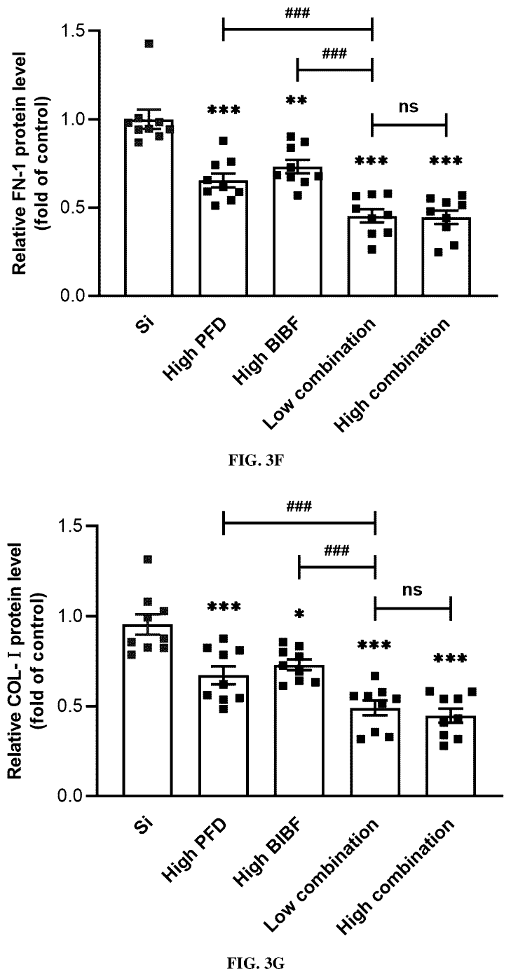

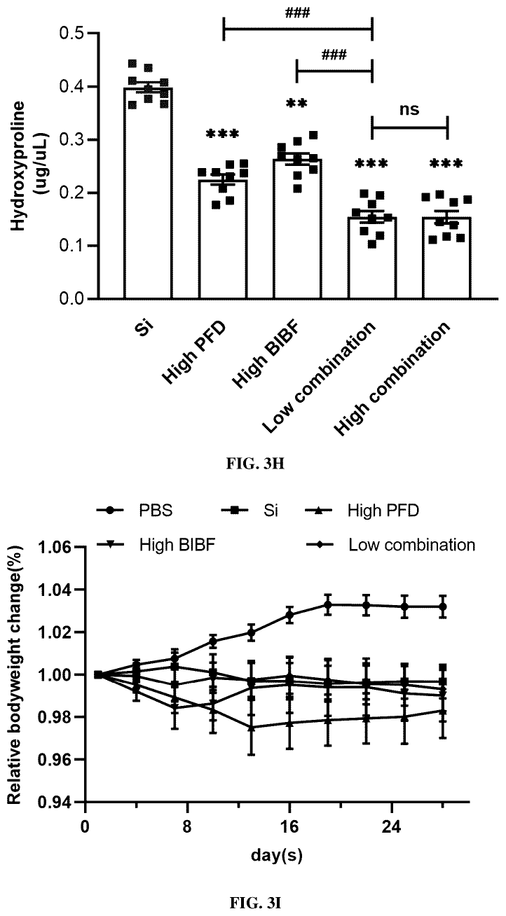

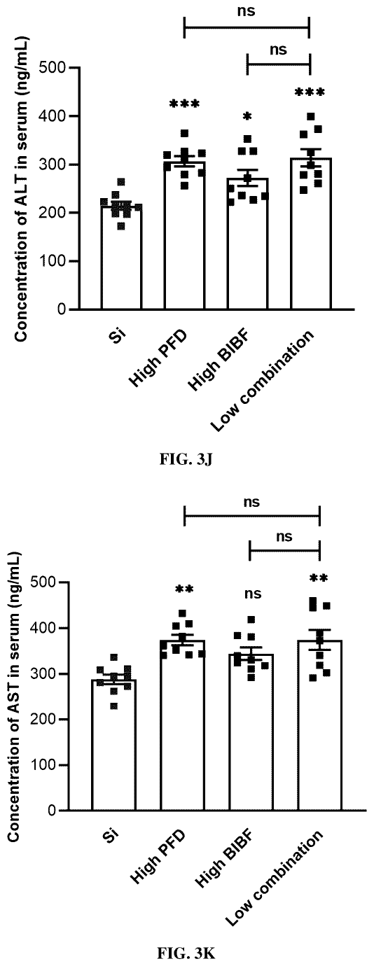

As shown in FIGS. 3A-3H, after the combined administration of PFD+BIBF 1120: the transcription level (FIG. 3C) and translation level (FIG. 3E and FIG. 3F) of the fibrosis factor FN-1 were reduced; transcription level (FIG. 3D) and translation level (FIG. 3E and FIG. 3G) of Col-I were reduced; the collagen-specific amino acid hydroxyproline (FIG. 3H) was reduced; and the Masson staining also showed that the fibrotic lesions were reduced (FIG. 3A and FIG. 3B) and the degree of the disease was alleviated.

In summary, combination therapy with PFD+BIBF 1120 showed superiority in terms of pulmonary function, inflammation, and fibrosis, and exhibited a better effect than therapy with PFD or BIBF 1120 alone. Meanwhile, after the combined administration of PFD and BIBF 1120, there was no significant change in the body weight of the mice (FIG. 3I), and there was also no significant increase in liver function injury indicators such as serum aminotransferase, ALT, and AST (FIG. 3J and FIG. 3K). The above results indicate that the combined treatment of PFD and BIBF 1120 has a desirable safety profile.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for treating pneumoconiosis, comprising administering a therapeutically effective amount of pirfenidone (PFD) combined with nintedanib (BIBF 1120).

2. The use according to claim 1, wherein the pneumoconiosis is one or more selected from the group consisting of silicosis, coal workers' pneumoconiosis (CWP), graphite pneumoconiosis, carbon black pneumoconiosis, asbestosis, talc pneumoconiosis, cement pneumoconiosis, mica pneumoconiosis, kaolin pneumoconiosis, aluminosis, arc-welders pneumoconiosis (AWP), and foundry workers' pneumoconiosis (FWP).

3. The use according to claim 1, wherein a PFD solution has a concentration of 10 mg/mL to 30 mg/mL.

4. The use according to claim 1, wherein a BIBF 1120 solution has a concentration of 2 mg/mL to 5 mg/mL.

5. The use according to claim 3, wherein the PFD solution or the BIBF 1120 solution is prepared with 1% carboxymethylcellulose sodium (CMC-Na) as a solvent.

6. A combined drug for treating pneumoconiosis, comprising PFD and BIBF 1120.

7. The combined drug according to claim 6, wherein PFD and BIBF 1120 are at a mass concentration ratio of 5 to 15:1.

8. The combined drug according to claim 6, further comprising a pharmaceutically acceptable carrier.

9. The combined drug according to claim 8, wherein the carrier comprises CMC-Na.

10. The combined drug according to claim 6, wherein a dosage form of the combined drug is selected from the group consisting of a powder, a tablet, a capsule, and a solution.

11. The use according to claim 5, wherein a BIBF 1120 solution has a concentration of 2 mg/mL to 5 mg/mL.


SEQUENCE LISTING (12 sequences)

SEQ ID NO: 1 - DNA, 18 bp, synthetic construct
  source 1..18: mol_type = other DNA; note = Upstream primer for beta-actin
taggcaccagggtgtgat

SEQ ID NO: 2 - DNA, 16 bp, synthetic construct
  source 1..16: mol_type = other DNA; note = Downstream primer for beta-actin
ctcctcaggggccaca

SEQ ID NO: 3 - DNA, 23 bp, synthetic construct
  source 1..23: mol_type = other DNA; note = Upstream primer for Fn-1
gacgaagagcccttacagttcca

SEQ ID NO: 4 - DNA, 20 bp, synthetic construct
  source 1..20: mol_type = other DNA; note = Downstream primer for Fn-1
tctgcagtgcctccactatg

SEQ ID NO: 5 - DNA, 19 bp, synthetic construct
  source 1..19: mol_type = other DNA; note = Upstream primer for Col-I
cctggtccctctggaaatg

SEQ ID NO: 6 - DNA, 20 bp, synthetic construct
  source 1..20: mol_type = other DNA; note = Downstream primer for Col-I
ggaagcctctttctcctctc

SEQ ID NO: 7 - DNA, 22 bp, synthetic construct
  source 1..22: mol_type = other DNA; note = Upstream primer for Il-1beta
caagcttccttgtgcaagtgtc

SEQ ID NO: 8 - DNA, 21 bp, synthetic construct
  source 1..21: mol_type = other DNA; note = Downstream primer for Il-1beta
ttcatcttttggggtccgtca

SEQ ID NO: 9 - DNA, 21 bp, synthetic construct
  source 1..21: mol_type = other DNA; note = Upstream primer for Il-6
ttcctctctgcaagagacttc

SEQ ID NO: 10 - DNA, 20 bp, synthetic construct
  source 1..20: mol_type = other DNA; note = Downstream primer for Il-6
gttgggagtggtatcctctg

SEQ ID NO: 11 - DNA, 20 bp, synthetic construct
  source 1..20: mol_type = other DNA; note = Upstream primer for Tnf-alpha
agacaccatgagcacagaaa

SEQ ID NO: 12 - DNA, 20 bp, synthetic construct
  source 1..20: mol_type = other DNA; note = Downstream primer for Tnf-alpha
cacttggtggtttgtgagtg